(19) United States
(12) Patent Application Publication
(10) Pub. No.: US 2022/0257980 A1
(43) Pub. Date: Aug. 18, 2022

(54) A METHOD AND A RADIOTHERAPY DEVICE FOR THERAPEUTIC ENERGY SPECTRUM CBCT

(71) Applicant: SUZHOU LINATECH MEDICAL SCIENCE AND TECHNOLOGY, Suzhou, Jiangsu (CN)

(72) Inventors: Hu Er WEN, Suzhou, Jiangsu (CN); Chunyan DAI, Suzhou, Jiangsu (CN); Jonathan Yi YAO, Suzhou, Jiangsu (CN)

(21) Appl. No.: 17/625,012

(22) PCT Filed: Nov. 11, 2019
(86) PCT No.: PCT/CN2019/117137

§ 371 Date: Jan. 5, 2022

(30) Foreign Application Priority Data
Jul. 9, 2019 (CN) 201910614155.7
Jul. 9, 2019 (CN) 201921068089.X

(51) Int. Cl. A61N 5/10; G01T 1/20

(52) U.S. Cl. CPC A61N 5/1049; A61B 6/035; A61N 2005/1061; G01T 1/20

(57) ABSTRACT

The present invention discloses an imaging device, method, and radiotherapy device for therapeutic energy spectrum CBCT. The device includes a double-layer detector and an image processing transmission device; the double-layer detector includes an upper layer detector and a lower layer detector; X-rays pass through the upper detector and are projected onto the lower detector; the upper detector is used to sense low energy X-rays and the lower detector is used to sense high energy X-rays. The image processing transmission device includes a first image processing transmission device and a second image processing transmission device. The first image processing transmission device corresponds to the upper detector and is used to process and transmit the sensing signal of the upper detector, while the second image processing transmission device corresponds to the lower detector and is used to process and transmit the sensing signal of the lower detector.

### TECHNICAL FIELD

The present invention relates to the field of medical technology, and specifically to an imaging device, method, and radiotherapy device for therapeutic energy spectrum CBCT.

### BACKGROUND

Cone Beam Computed Tomography (CBCT) for radiotherapy is the most widely used image guidance technique in the field of radiation therapy. It generally uses a large-area amorphous silicon digital X-ray detector plate, and the accelerator rack rotates for a cycle or a large half cycle to acquire and reconstruct the electronic computed tomography (CT) images within a certain volume, mainly for position verification during radiotherapy placement and radiation treatment to guide the current treatment and/or subsequent fractional treatment.

However, the image quality of CBCT for radiotherapy is poor due to the presence of radiographic artifacts, low density resolution (especially low contrast density resolution), and other reasons. Artifacts in cone-beam images are mainly due to X-ray scattering, the motion of the scanned object, and the performance of the flat panel detector itself. The scattered signal from the center of the detector produces a “cupping” artifact in the projected image; compared to sector beam CT, the scattering of X-rays reaching the CBCT detector plate is significantly higher, which has a more pronounced effect on image quality. In addition, the hardening artifact of sector beam CT is also difficult to eliminate in cone-beam CT. The so-called X-ray hardening refers to the fact that the spherical tube produces a mixture of X-rays, and the illuminated material is selective for X-rays of different energies, and generally, lower-energy X-rays are absorbed more, so the X-rays “harden” after passing through the illuminated material.

### SUMMARY

The purpose of the present invention is to provide an imaging device, method, and radiotherapy device for therapeutic energy spectrum CBCT to address the problem of poor image quality of CBCT in response to the above-mentioned deficiencies in the prior art.

To achieve the above purpose, the technical solutions used in the present invention are as follows:

Firstly, the present invention provides an imaging device for therapeutic energy spectrum CBCT, the device includes: a double-layer detector and an image processing transmission device, the double-layer detector comprising an upper layer detector and a lower layer detector, the X-rays from the X-ray emitting device passing through the upper layer detector and irradiating to the lower layer detector, the upper layer detector is used to sense low energy X-rays, the lower layer detector is used to sense high energy X-rays, the image processing transmission device comprising a first image processing transmission device and a second image processing transmission device, the first image processing transmission device is provided in correspondence with the upper detector and is used to process and transmit the sensing signal of the upper detector, the second image processing transmission device is provided in correspondence with the lower detector and is used to process and transmit the sensing signal of the lower detector.

Optionally, the device further comprises a filter between the upper detector and the lower detector; the filter is used to filter out the low energy X-rays in the X-rays after passing through the upper detector.

Optionally, the device further comprises a motion control device, which is used to control the overall motion of the double-layer detector, the image processing transmission device, and the filter.

Optionally, the shape of the double-layer detector is flat or curved-shaped. Optionally, the scintillator of the upper detector is made of ZnSe or CS/and the scintillator of the lower detector is made of Gd₂O₂S.

Optionally, the material comprising the filter includes at least one of: air, copper, titanium, aluminum, iodine, gadolinium.

Optionally, the structure of the filter is a circular structure or a square structure formed by a single material or a mixture of multiple materials.

Optionally, the structure of the filter is a tessellated structure or a concentric circle structure formed by a staggered arrangement of different materials in a plurality of materials.

Secondly, the invention also provides a method for therapeutic energy spectrum CBCT, the method is according to an apparatus as described in the first aspect above, the method comprising:

switching on an X-ray emitting device and a double-layer detector;

acquiring a low energy X-ray image by the upper detector;

acquiring a high energy X-ray image by the lower detector;

performing an energy spectrum CBCT image reconstruction using the low energy X-ray image and the high-energy X-ray image to obtain a tomographic image.

Thirdly, the invention also provides a radiotherapy device, the radiotherapy device comprising an imaging device for therapeutic energy spectrum CBCT described in the first aspect, or using a method for therapeutic energy spectrum CBCT described in the second aspect to perform imaging.

The beneficial effects of the present invention include:

The imaging device for therapeutic energy spectrum CBCT proposed in the present invention includes: a double-layer detector and an image processing transmission device, the double-layer detector comprising an upper layer detector and a lower layer detector, the X-rays from the X-ray emitting device pass through the upper layer detector and are irradiated to the lower layer detector, the upper layer detector is used to sense low energy X-rays and the lower layer detector is used to sense high energy X-rays. The image processing transmission device comprising a first image processing transmission device and a second image processing transmission device, the first image processing transmission device corresponding to the upper detector and used to process and transmit the sensing signal of the upper detector, and the second image processing transmission device corresponding to the lower detector and used to process and transmit the sensing signal of the lower detector. By using the upper detector that senses low energy X-rays and the lower detector that senses high energy X-rays respectively, it is possible to achieve energy spectrum imaging, which can effectively remove radiographic artifacts and provide rich anatomical information, thus improving the image quality of CBCT.

### BRIEF DESCRIPTION OF THE DRAWINGS

In order to illustrate the technical solutions of the embodiments of the present invention more clearly, the following drawings are briefly described for use in the embodiments. It should be understood that the following drawings only illustrate certain embodiments of the present invention and therefore should not be regarded as limiting the scope, and that other relevant drawing may be obtained on the basis of these drawings without creative effort by a person of ordinary skill in the art.

FIG. 1 illustrates a schematic diagram of the structure of a CBCT on a conventional accelerator.

FIG. 2 illustrates a schematic diagram of the structure of an imaging device for therapeutic energy spectrum CBCT provided in one embodiment of the present invention

FIG. 3 illustrates a schematic diagram of the structure of an imaging device for therapeutic energy spectrum CBCT provided by another embodiment of the present invention

FIG. 4A and FIG. 4B illustrate schematic diagrams of the structure of a double-layer detector provided by embodiments of the present invention

FIG. 5A, FIG. 5B, FIG. 5C and FIG. 5D illustrates a schematic diagram of the structure of a filter provided by an embodiment of the present invention

FIG. 6A and FIG. 6B illustrate schematic diagrams of the opening method of the imaging device provided by embodiments of the present invention

FIG. 7 illustrates a schematic diagram of the direction of movement of the imaging device provided by embodiments of the present invention; and

FIG. 8 illustrates a schematic diagram of the flow of the imaging method of radiotherapy energy spectrum CBCT provided by embodiments of the present invention.

The accompanying markings: 101—X-ray bulb; 102—flat panel detector; 200—double-layer detector; 201—upper detector; 202—lower detector; 203—first image processing transmission device; 204—second image processing transmission device; 205—object to be measured; 206—accelerator treatment bed; 207—electron beam; 208—anode target; 209—filter 210—motion control device; 700—imaging device.

### DETAILED DESCRIPTION OF THE EMBODIMENTS

The technical solutions in the embodiments of the present invention will be clearly and completely described below in conjunction with the accompanying drawings in the embodiments of the present invention, and it is clear that the described embodiments are only a part of the embodiments of the present invention, and not all of them. Based on the embodiments in the present invention, all other embodiments obtained by a person of ordinary skill in the art without making creative labor fall within the scope of protection of the present invention.

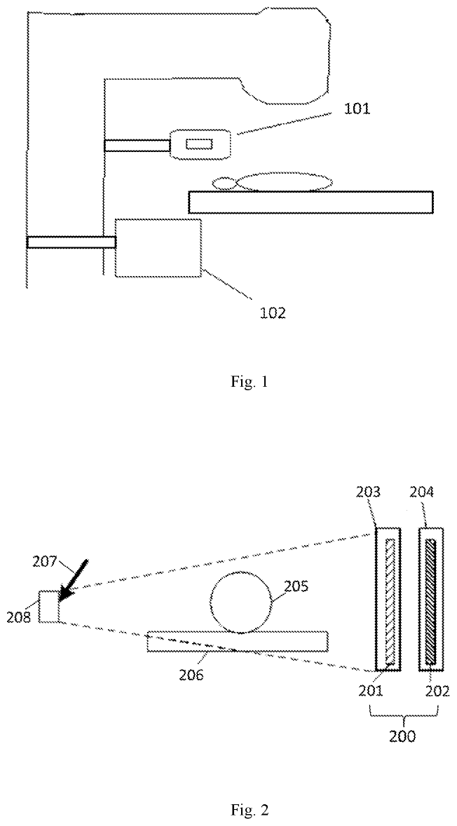

As shown in FIG. 1, a conventional radiotherapy CBCT device is mounted in the direction of the radiotherapy accelerator gantry perpendicular to the radiotherapy beam and includes an X-ray bulb 101 and a flat panel detector 102. The installation position of the therapeutic energy spectrum CBCT double-layer detector system provided by the present invention is located at the position of the flat panel detector 102 to replace the conventional flat panel detector 102.

FIG. 2 illustrates a schematic diagram of the structure of an imaging device for therapeutic energy spectrum CBCT provided in an embodiment of the present invention, as shown in FIG. 2, the device includes a double-layer detector 200 and an image processing transmission device, the double-layer detector 200 includes an upper layer detector 201 and a lower layer detector 202; the object to be measured 205 located on the accelerator treatment bed 206 is between the X-ray emitting device and the double-layer detector 200, and X-rays from the X-ray emitting device (e.g., X-rays generated by the electron beam 207 bombarding the anode target 208 shown in FIG. 2) pass through the upper layer detector 201 and are irradiated to the lower layer detector 202. The upper detector 201 is used for sensing low energy X-rays and the lower detector 202 is used for sensing high energy X-rays, and the image processing transmission device includes a first image processing transmission device 203 and a second image processing transmission device 204, with the first image processing transmission device 203 corresponding to the upper detector 201 and used for processing and transmitting the sensing signal of the upper detector 201, and the second image processing transmission device 204 corresponding to the lower detector 202 and used for processing and transmitting the sensing signal of the lower detector 202.

In the imaging process, the X-rays passing through the object to be measured 205 first reach the upper detector 201, which absorbs and senses the low energy X-rays in the X-rays, and the high energy X-rays not absorbed by the upper detector 201 pass through the upper detector 201 and reach the lower detector 202, which are thus absorbed and sensed by the lower detector 202.

In summary, by using the upper detector that senses low energy X-rays and the lower detector that senses high energy X-rays respectively, energy spectrum imaging can be achieved, which can effectively remove the artifacts and provide rich anatomical information, thus improving the image quality of CBCT.

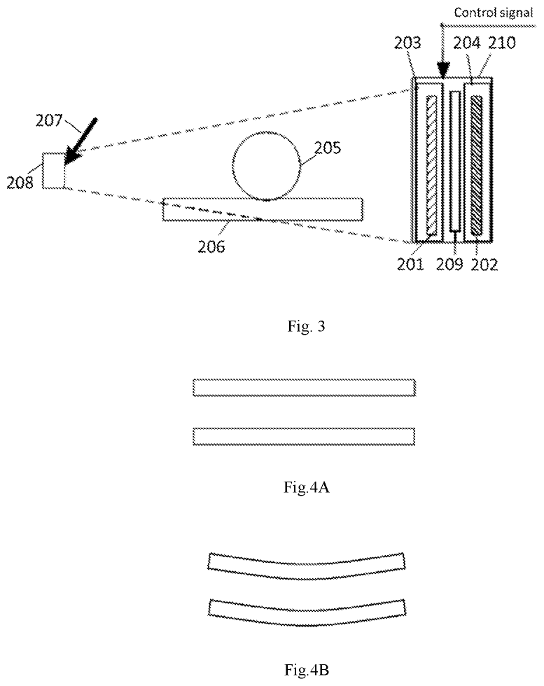

Optionally, as shown in FIG. 3, the imaging device provided by the present invention further comprises a filter 209 provided between the upper detector 201 and the lower detector 202, the filter 209 is used to filter out the low energy X-rays from the X-rays after passing through the upper detector 201. By providing a predetermined thickness of the filter 209, the energy spectrum of the X-rays can be adjusted after passing through the upper detector 201, and the energy spectrum of the X-rays can be further differentiated before the lower detector 202 is imaged, thereby improving the imaging quality of the lower detector 202. The thickness of the filter 209 is adjustable and can be adjusted according to the actual material of the filter 209 and in combination with the X-ray energy spectrum.

Optionally, the imaging device provided by the present invention also includes a motion control device 210, the control end of the motion control device 210 can receive digital or analog control signals, and the motion control device 210 responds to the control signals to control the overall movement of the entire imaging device including the double-layer detector 200, image processing transmission device (including the first image processing transmission device 203 and the second image processing transmission device 204) and filter 209. Specifically, the motion control device 210 can include a drive mechanism and a control mechanism, where the drive mechanism is made of piezoelectric material, and the control mechanism can receive control signals and process them into control levels to make the drive mechanism drive the entire imaging device system movement according to the actual demand. The control signal can be a digital signal or an analog signal such as pulse, sine wave, etc. By setting up the motion control device 210, the whole detector system is made movable to solve the problem of small imaging range that may occur in the clinic.

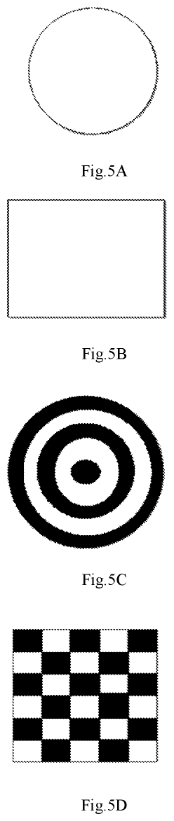

As shown in FIG. 4A and FIG. 4B, the shape of the double-layer detector 200 can be flat-plate or curved. In other words, the structure of the double-layer detector 200 can be a double-layer flat detector (FIG. 4A) or a double-layer curved detector with a certain degree of arc (FIG. 4B). For example, the double-layer flat panel detector can be used in the embodiment of the invention, and the double-layer flat panel detector can be formed by setting two conventional single-layer flat panel detectors, of which the single-layer flat panel detector is widely used and the technology is more mature.

For the double-layer detector 200 provided by embodiments of the present invention, by design, the double-layer detector 200 is a scintillator of two different sensitivities, the upper layer being sensitive to low energy X-rays and the lower layer being sensitive to high energy X-rays. Specifically, the material of the scintillator of the upper detector 201 may be a multiple composite material, for example, it may be ZnSe or CSI. The material of the scintillator of the lower detector 202 can be a multiple composite material, for example, it can be Gd₂O₂S. And with the addition of filter 209, the choice of scintillator material of the lower detector 202 is more extensive.

The material comprising the filter 209 is a ray-filtering material, including, for example, at least one of the following: air, copper, titanium, aluminum, iodine, gadolinium.

As shown in FIG. 5A to FIG. 5D, the filter 209 can be a structure made of a single material or a mixture of materials, for example, the structure of the filter 209 can be a circular structure (FIG. 5A) or a square structure (FIG. 5B) formed by a single material or a mixture of materials. The structure of the filter 209 can also be a structure made of two or more materials, for example, the structure of the filter 209 can be a tessellated structure (FIG. 5D) or a concentric circle structure (FIG. 5C) formed by the interlacing arrangement of two different materials. For example, a square copper sheet can be used as the structure of the filter 209, which has the advantages of simple process, easy access to materials, better ductility, and the ability to adjust its thickness according to the actual situation. The design of filter 209 can increase the differentiation of the energy spectrum of the upper and lower detectors, effectively solving the impact of artifacts and the lack of rich anatomical information on the image quality.

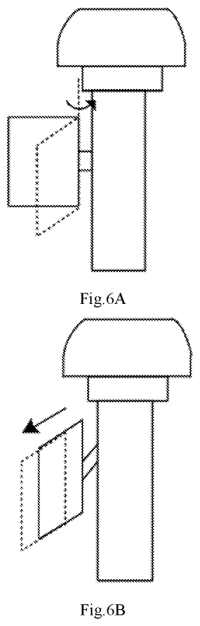

FIG. 6A and FIG. 6B illustrate schematic diagrams of the opening method of the imaging device provided by embodiments of the present invention. For example, the imaging device can be opened in a rotating (FIG. 6A) or telescoping (FIG. 6B) manner.

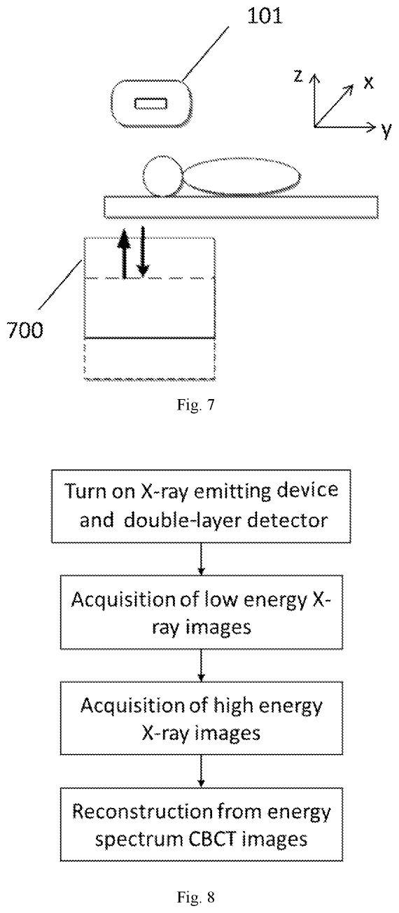

As shown in FIG. 7, the movable direction of the imaging device 700 in the embodiment of the present invention is the z-direction of the three-dimensional coordinates of the accelerator. The imaging device 700 may be any of the imaging devices provided in the above embodiments of the present invention. By the movement of the imaging device 700, the imaging range of the object on the imaging plate can be increased.

Embodiments of the present invention also provide an imaging method for therapeutic energy spectrum CBCT, which is used in the imaging device provided in the above embodiments of the present invention.

As shown in FIG. 8, the X-ray emitting device and the double-layer detector system are first turned on by motion control signals, and images are acquired when the X-rays are exposed, with the upper detector acquiring low energy X-ray images and the lower detector acquiring high-energy X-ray images, and two sets of energy spectrum CBCT images are obtained after the acquisition is completed; then energy spectrum CBCT image reconstruction is performed to obtain and display tomographic images. If it is found that the range of the physically acquired images under test is not enough, the double-layer detector can also be moved by the motion control device after the acquisition is completed to obtain two more sets of energy spectrum CBCT images.

In addition, the present invention provides a radiotherapy device, the radiotherapy device comprising an imaging device for therapeutic energy spectrum CBCT as provided above in the present invention, or an imaging method for therapeutic energy spectrum CBCT as provided above in the present invention is used for imaging.

The above embodiments are only to illustrate the technical conception and features of the present invention and are intended to enable a person of ordinary skill in the art to understand the content of the present invention and to implement it, and not to limit the scope of protection of the present invention in this way, and any equivalent changes or modifications made according to the spirit of the present invention shall be covered within the scope of protection of the present invention.

1. An imaging device for therapeutic energy spectrum CBCT includes: a double-layer detector and an image processing transmission device; the double-layer detector comprising an upper layer detector and a lower layer detector, X-rays from an X-ray emitting device passing through the upper layer detector and irradiating to the lower layer detector, the upper layer detector is used to sense low energy X-rays and the lower layer detector is used to sense high energy X-rays, the image processing transmission device comprising a first image processing transmission device and a second image processing transmission device, the first image processing transmission device being provided in correspondence with the upper detector and for processing and transmitting the sensing signal of the upper detector, the second image processing transmission device being provided in correspondence with the lower detector and for processing and transmitting the sensing signal of the lower detector.

2. The device according to claim 1, wherein it further comprises a filter arranged between the upper detector and the lower detector, and the filter is used to filter low energy X-rays in the X-rays after passing through the upper detector.

3. The device according to claim 2, wherein it further comprises a motion control device, the motion control device for controlling the overall motion of the double-layer detector, the image processing transmission device, and the filter.

4. The device according to claim 1, wherein the double-layer detector has a flat or curved shape.

5. The device according to claim 4, wherein the material of the scintillator of the upper detector is ZnSe or CSI, and the material of the scintillator of the lower detector is Gd₂O₂S.

6. The device according to claim 2, wherein the material constituting the filter comprises at least one of air, copper, titanium, aluminum, iodine, gadolinium.

7. The device according to claim 6, wherein the structure of the filter is a circular structure or a square structure formed by a single material or a mixture of multiple materials.

8. The device according to claim 6, wherein the structure of the filter is a tessellated structure or a concentric circle structure formed by a staggered arrangement of different materials in a plurality of materials.

9. A method for therapeutic energy spectrum CBCT, the method according to claim 1, the method comprising:
switching on an X-ray emitting device and a double-layer detector;
acquiring a low-energy X-ray image by the upper detector;
acquiring a high-energy X-ray image by the lower detector;
performing an energy spectrum CBCT image reconstruction using the low-energy X-ray image and the high-energy X-ray image to obtain a tomographic image.

10. A radiotherapy device, the radiotherapy device comprises an imaging device for therapeutic energy spectrum CBCT according to claim 1.
